(19) United States
(12) Patent Application Publication
(10) Pub. No.: US 2026/0013949 A1
(43) Pub. Date: Jan. 15, 2026

(54) APPARATUS FOR TRACING A CATHETER

(71) Applicant: KONINKLIJKE PHILIPS N.V., EINDHOVEN (NL)

(72) Inventors: ILYAS SIRAZITDINOV, HAMBURG (DE); JOËL VALENTIN STADELMANN, MOSCOW (RU); AXEL SAALBACH, HAMBURG (DE); HEINRICH SCHULZ, HAMBURG (DE)

(21) Appl. No.: 18/881,341

(22) PCT Filed: Jun. 26, 2023
(86) PCT No.: PCT/EP2023/067216

§ 371 Date: Jan. 6, 2025

(30) Foreign Application Priority Data
Jul. 7, 2022 (RU) 2022118588

(51) Int. Cl. A61M 25/01; A61B 34/20; G06T 3/18; G06T 7/70

(52) U.S. Cl. CPC A61M 25/0105; G06T 3/18; G06T 7/70; A61B 2034/2065; A61M 2025/0166; A61M 2205/502

(57) ABSTRACT

The present invention relates to an apparatus (10) for tracing a catheter or tube, comprising: an input unit (20); and a processing unit (30). The input unit is configured to receive a medical image of a patient. The input unit is configured to provide the medical image of the patient to the processing unit. The input unit is configured to receive a live data stream from one or more sensors, wherein the live data stream comprises data of the patient, and wherein a sensor coordinate system is defined with respect to the one or more sensors. The input unit is configured to provide the live data stream to the processing unit. The processing unit is configured to receive a sensed location of an end of a catheter or tube that is to be inserted into the patient. The processing unit is configured to align the data of the patient received from the one or more sensors with the sensor coordinate system. The processing unit is configured to map the medical image of the patient to the sensor coordinate system. The processing unit is configured to determine a location of the end of the catheter or tube in the sensor coordinate system comprising utilization of the sensed location of the end of the catheter or tube. The processing unit is configured to generate a representation of the sensed location of the end of the catheter or tube with respect to the medical image comprising utilization of the medical image of the patient mapped to the sensor coordinate system and the location of the end of the catheter or tube in the sensor coordinate system.

### FIELD OF THE INVENTION

The present invention relates to an apparatus for tracing a catheter, a system for tracing a catheter, a method for tracing a catheter, as well as to a computer program element and a computer readable medium.

### BACKGROUND OF THE INVENTION

Chest X-ray (CXR) is a one of the most common examinations in radiology departments due to its relatively low cost and small acquisition time. As a result, radiology departments can overflow with CXRs that can drastically increase report turn-around times.

CXR is often used as the main modality to assess position of foreign objects, e.g. central venous catheter (CVC) (see for example Pikwer A, Bååth L, Davidson B, Perstoft I, Akeson (2010). The incidence and risk of central venous catheter malpositioning: A prospective cohort study in 1619 patients. Anaesth Intensive Care. 2008; 36:30-7, and Yi, X., Adams, S. J., Henderson, R. D., & Babyn, P. (2020). Computer-aided Assessment of Catheters and Tubes on Radiographs: How Good Is Artificial Intelligence for Assessment? Radiology: Artificial Intelligence, 2 (1), e190082), endotracheal tube (see for example Chen, S., Zhang, M., Yao, L., & Xu, W. (2016). Endotracheal tubes positioning detection in adult portable chest radiography for intensive care unit. International journal of computer assisted radiology and surgery, 11 (11), 2049-2057), and feeding tube (see for example Singh, V., Danda, V., Gorniak, R., Flanders, A., & Lakhani, P. (2019). Assessment of critical feeding tube malpositions on radiographs using deep learning. Journal of digital imaging, 32 (4), 651-655), etc. Precise and right-time detection of malpositioned foreign objects can be very important for particular external objects, for example the insertion of a CVC is notoriously difficult and could result in a pneumothorax (PTX). In addition to the initial positioning of the device, daily verification using X-ray imaging is commonly performed. The incidence of a CVC malposition ranges from 3.6 to 14% (see: Pikwer A, Bååth L, Davidson B, Perstoft I, Akeson (2010). The incidence and risk of central venous catheter malpositioning: A prospective cohort study in 1619 patients. Anaesth Intensive Care. 2008; 36:30-7). Philips' clinical partner the University Hospital Hamburg-Eppendorf reported that currently 20% of all CXRs have this clinical question. Conventionally, catheters and tubes are inserted in a blind way or with a help of ultra-sound guidance. The placement of the device in this manner is then usually verified after the insertion procedure on one or CXR, which as described above can lead to too many CXRs requiring to be performed and increased report turn-around times

Hence, there is a clinical need of automatic solutions that can reduce the workload of a radiologist in a hospital.

### SUMMARY OF THE INVENTION

It would be advantageous to provide an improved technique to facilitate the reduction in the need for X-ray examinations relating to the insertion of catheters and tubes into patients.

The object of the present invention is solved with the subject matter of the independent claims, wherein further embodiments are incorporated in the dependent claims. It should be noted that the following described aspects and examples of the invention apply to the apparatus for tracing a catheter, the system for tracing a catheter, the method for tracing a catheter, as well as to a computer program element and a computer readable medium,

In a first aspect, there is provided a n apparatus for tracing a catheter or tube, comprising:
an input unit; and
a processing unit.

The input unit is configured to receive a medical image of a patient. The input unit is configured to provide the medical image of the patient to the processing unit. The input unit is configured to receive a live data stream from one or more sensors. The live data stream comprises data of the patient, and a sensor coordinate system is defined with respect to the one or more sensors. The input unit is configured to provide the live data stream to the processing unit. The processing unit is configured to receive a sensed location of an end of a catheter or tube that is to be inserted into the patient. The processing unit is configured to align the data of the patient received from the one or more sensors with the sensor coordinate system. The processing unit is configured to map the medical image of the patient to the sensor coordinate system. The processing unit is configured to determine a location of the end of the catheter or tube in the sensor coordinate system comprising utilization of the sensed location of the end of the catheter or tube. The processing unit is configured to generate a representation of the sensed location of the end of the catheter or tube with respect to the medical image comprising utilization of the medical image of the patient mapped to the sensor coordinate system and the location of the end of the catheter or tube in the sensor coordinate system.

In other words, a previous medical image of a patient is provided that could have been taken a year ago, a month ago, a week ago, a day ago, an hour ago or a few minutes ago. The medical image will show details such as the anatomical structure of the patient, such as for example the vein structure of the patient. The patient is now needing to have a catheter or tube or tubus inserted into their body, for example a central venous catheter (CVC) is to be inserted into their body. However, the insertion of a CVC is very difficult, and CVCs are frequently malpositioned.

Therefore, a new technique has been developed to register a medical image of the patient with or in effect on the patient, Thus an X-ray image or MR image (or other medical image), that has been previously acquired, is registered or aligned with the patient in order that as a medical practitioner is inserting a catheter or tube into the patient they are provided via the medical image with an anatomical structure of the patient, that has in effect been mapped to the patient in front of them, in order that they can correctly insert the catheter into the correct anatomical structure, such as a vein, that they can see via the medical image that has been registered with the patient.

The new technique uses data from a sensor system, such as a camera system, lidar system, radar system, that shows the patient. The sensor system can use anchors, antennas, or other markers in the room where the patient is located to generate a sensor coordinate system. The sensor system also provides data of the patient, such as image data and/or depth data and/or radar data in a real time data stream and places the data of the patient in the real time data stream into the sensor coordinate system. The previously acquired medical image of the patient is then mapped to the sensor coordinate system via image registration. A location of the end of the catheter or tube to be inserted into the patient is then sensed and also placed into sensor coordinate system. As both the medical image and the location of the end of the catheter or tube are aligned with the sensor coordinate system, the location of the end of the catheter or tube can be represented with respect to the medical image. Thus, the medical image, or a warped medical image, can be presented on a visual display unit along with the location of the end of the catheter or tube. Then as a medical expert moves the catheter or tube towards the patient the end of the tube or catheter can be represented as moving with respect to the medical image on the visual display unit. The medical expert can then see the relevant anatomical structure, e.g. vein structure, of the patient, for example, and can insert the catheter or tube correctly. As the medical image is mapped to the live data stream as the patient moves the medical image of the patient can be represented to account for that movement, such that the medical expert has a continually correct representation of for example the vein structure of the patient to be viewed and can ensure correct insertion of the catheter or tube.

In an example, the processing unit is configured to generate a mapped medical image of the patient comprising utilization of the medical image of the patient mapped to the sensor coordinate system. The generation of the representation of the sensed location of the end of the catheter or tube with respect to the medical image comprises a generation of the representation of the sensed location of the end of the catheter or tube in the mapped medical image of the patient.

Thus, the medical image, such as an X-ray image or MR image, is warped to match the data of the patient acquired by one or more sensors, such as image data (that defines the sensor coordinate system). Thus, if the medical image was acquired 2 months ago when the patient was heavier than they are now, this medical image is warped to match how the patient now looks as sensed by the one or more sensors.

In an example, the processing unit is configured to generate a modified sensor coordinate system comprising utilization of the medical image of the patient mapped to the sensor coordinate system. The determination of the location of the end of the catheter or tube in the sensor coordinate system then comprises a determination of the location of the end of the catheter or tube in the modified sensor coordinate system. The generation of the representation of the sensed location of the end of the catheter or tube with respect to the medical image then comprises a generation of the representation of the sensed location of the end of the catheter or tube in the medical image of the patient.

In other words, the medical image of the patient forms ground truth data and is not warped, but when aligning the medical image to the sensor coordinate system the sensor coordinate system is itself warped, such that in effect the live data feed of the patient becomes warped. The medical image is therefore always realistic and not warped but the location of the end of the catheter or tube is now presented with the warped sensor coordinate system and can then move slightly differently to the movement the medical expert makes, because for example the patient can move, but the medical expert is always presented with an unwarped version or the medical image of the patient with an unwarped vein structure for example.

To put this another way, the data of the patient, such as image data, acquired by the one or more sensors (that defines the sensor coordinate system) is warped to match the medical image, such as an X-ray image or MR image that could have been acquired several month ago when the patient was heavier than they are now. Thus, the medical image is in effect ground truth and the space where the patient is located is in effect warped. Thus, as before if the patient was heavier when the medical image was acquired, the medical professional will see this image on their screen. This image is bigger than the patient is at the moment, however the representation of space is warped such that as the catheter is moved towards the patient it reaches the patient and the X-ray image at the same time and at the same point, and at a correct point as indicated by the medical image.

It can be useful to switch to an unwarped medical image being presented within a warped sensor coordinate system to verify the anatomical structure of the patient, such as a vein structure, before switching back to the warped medical image for the actual insertion into the patient of the catheter or tube or vice versa.

In an example, the one or more sensors comprises a plurality of sensors, and wherein the live data stream comprises data from each of the plurality of sensors.

In an example, the alignment of the data of the patient with the sensor coordinate system comprises utilization of data of the patient from each of the plurality of sensors.

Thus, for example image data from a 3D camera or radar or lidar sensor can on its own determine the location of the patient with respect to the location of such sensors and place the patient in the sensor coordinate system. However, a number of sensors can be utilized to generate this information. Thus, for example a number of cameras can in effect provide 3D data of the patient, or a number of radar systems can provide range and direction information to provide detailed location information of the patient.

Thus triangulation, markers, antennas can be used to locate the data of the patient, that is in effect 3D data, within the sensor coordinate system.

In an example, the live data stream comprises data of the end of the catheter or tube, and wherein the sensed location of the end of the catheter or tube that is to be inserted into the patient is determined from the data of the end of the catheter or tube from each of the plurality of sensors.

Thus for example, triangulation can be used to sense the location of the end of the catheter or tube accurately within the sensor coordinate system.

In an example, a radiolocation system is configured to sense the location of the end of the catheter or tube, and wherein the radiolocation system is configured to provide the sensed location of the end of the catheter or tube to the processing unit.

In other words, a radar system is used to sense the end of the catheter or tube. The radar system is at a known location and orientation within the camera coordinate system and the sensed location of the end of the catheter or tube with respect to the radar system is then known with respect to the camera coordinate system.

In an example, the catheter or tube comprises a fiber optic shape sensing system configured to sense the location of the end of the catheter or tube. The fiber optic shape sensing system is configured to provide the sensed location of the end of the catheter or tube to the processing unit.

In an example, mapping the medical image of the patient to the sensor coordinate system comprises registration between the medical image of the patient and at least one frame of the live data stream.

Thus for example, a frame of the data stream can be matched to the medical image, which can involve finding an applying a warping transformation that aligns the data of the patient's body in the data frame with the medical image of the patient, and where this can be achieved using classical computer vision algorithms or by neural networks.

Thus, a camera image can be mapped to the medical image (e.g. X-ray image or MR image), or a radar image can be mapped to the medical image (e.g. X-ray image or MR image) or depth data from a radar/RGB-D camera/lidar can be mapped to the medical image (e.g. X-ray image or MR image).

In an example, registration between the medical image of the patient and at least one frame of the live data stream comprises utilization of a warping transformation to align the medical image of the patient body with the data of the patient in the at least one frame of the live data stream.

In a second aspect, there is provided an system for tracing a catheter or tube, comprising:
an input unit;
a processing unit;
one or more sensors; and
a visual display unit.
   The input unit is configured to receive a medical image of a patient. The input unit is configured to provide the medical image of the patient to the processing unit. The input unit is configured to receive a live data stream from the one or more sensors. The live data stream comprises data of the patient, and a sensor coordinate system is defined with respect to the one or more sensors. The input unit is configured to provide the live data stream to the processing unit. The processing unit is configured to receive a sensed location of an end of a catheter or tube that is to be inserted into the patient. The processing unit is configured to align the data of the patient received from the one or more sensors with the sensor coordinate system. The processing unit is configured to map the medical image of the patient to the sensor coordinate system. The processing unit is configured to determine a location of the end of the catheter or tube in the sensor coordinate system comprising utilization of the sensed location of the end of the catheter or tube. The processing unit is configured to generate a representation of the sensed location of the end of the catheter or tube with respect to the medical image on the visual display unit comprising utilization of the medical image of the patient mapped to the sensor coordinate system and the location of the end of the catheter or tube in the sensor coordinate system.

In an example, the processing unit is configured to generate a mapped medical image of the patient comprising utilization of the medical image of the patient mapped to the sensor coordinate system. The processing unit is configured to present the mapped medical image on the visual display unit. The generation of the representation of the sensed location of the end of the catheter or tube with respect to the medical image then comprises a generation of the representation of the sensed location of the end of the catheter or tube in the mapped medical image of the patient presented on the visual display unit.

In an example, the processing unit is configured to generate a modified sensor coordinate system comprising utilization of the medical image of the patient mapped to the sensor coordinate system. The determination of the location of the end of the catheter or tube in the sensor coordinate system then comprises a determination of the location of the end of the catheter or tube in the modified sensor coordinate system. The processing unit is configured to present the medical image on the visual display unit. The generation of the representation of the sensed location of the end of the catheter or tube with respect to the medical image then comprises a generation of the representation of the sensed location of the end of the catheter or tube in the medical image of the patient presented on the visual display unit.

In a third aspect, there is provided a method for tracing a catheter or tube, comprising:
receiving a medical image of a patient;
providing the medical image of the patient to a processing unit;
receiving a live data stream from one or more sensors, wherein the live data stream comprises data of the patient, and wherein a sensor coordinate system is defined with respect to the one or more sensors;
providing the live data stream to the processing unit;
receiving by the processing unit a sensed location of an end of a catheter or tube that is to be inserted into the patient;
aligning by the processing unit the data of the patient received from the one or more sensors with the sensor coordinate system;
mapping by the processing unit the medical image of the patient to the sensor coordinate system;
determining by the processing unit a location of the end of the catheter or tube in the sensor coordinate system comprising utilizing the sensed location of the end of the catheter or tube; and
generating by the processing unit a representation of the sensed location of the end of the catheter or tube with respect to the medical image comprising utilizing the medical image of the patient mapped to the sensor coordinate system and the location of the end of the catheter or tube in the sensor coordinate system.

According to aspects, there is provided computer program elements controlling one or more of the apparatuses and/or systems as previously described which, if the computer program element is executed by a processor, is adapted to perform the method as previously described.

According to other aspects, there is provided computer readable media having stored the computer elements as previously described.

The computer program element can for example be a software program but can also be a FPGA, a PLD or any other appropriate digital means.

Advantageously, the benefits provided by any of the above aspects equally apply to all of the other aspects and vice versa.

The above aspects and examples will become apparent from and be elucidated with reference to the embodiments described hereinafter.

### BRIEF DESCRIPTION OF THE DRAWINGS

Exemplary embodiments will be described in the following with reference to the following drawings:

FIG. 1 shows a schematic representation of an apparatus for tracing a catheter;

FIG. 2 shows a schematic representation of a system for tracing a catheter;

FIG. 3 shows a method for tracing a catheter;

FIG. 4 shows on the left the anatomical overview and important areas for a CVC, where each of the four veins can be used to insert a CVC, and shows on the right a demonstration of a correctly positioned CVC after insertion; and

FIG. 5 shows a schematic representation of a detailed embodiment of a system for tracing a catheter.

### DETAILED DESCRIPTION OF EMBODIMENTS

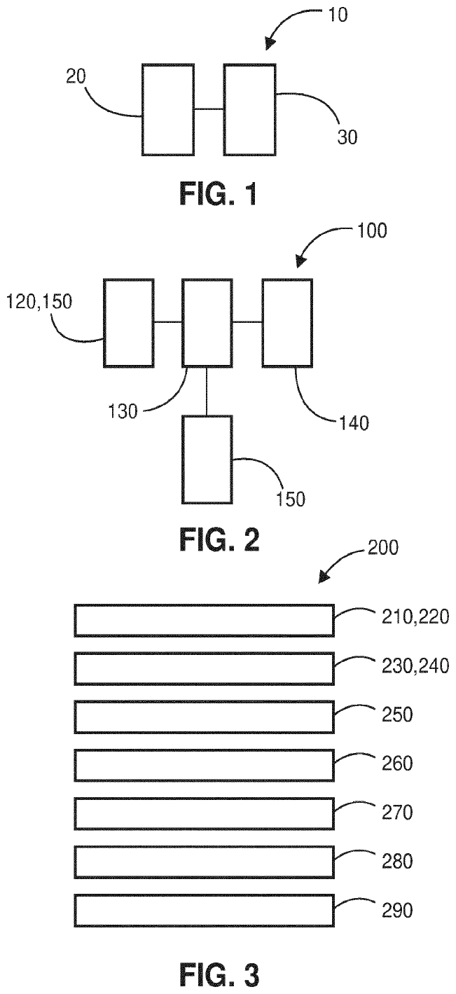

FIG. 1 shows an example of an apparatus 10 for tracing a catheter or tube. The apparatus comprises an input unit 20, and a processing unit 30. The input unit is configured to receive a medical image of a patient. The input unit is configured to provide the medical image of the patient to the processing unit. The input unit is configured to receive a live data stream from one or more sensors. The live data stream comprises data of the patient, and a sensor coordinate system is defined with respect to the one or more sensors. The input unit is configured to provide the live data stream to the processing unit. The processing unit is configured to receive a sensed location of an end of a catheter or tube that is to be inserted into the patient. The processing unit is configured to align the data of the patient received from the one or more sensors with the sensor coordinate system. The processing unit is configured to map the medical image of the patient to the sensor coordinate system. The processing unit is configured to determine a location of the end of the catheter or tube in the sensor coordinate system comprising utilization of the sensed location of the end of the catheter or tube. The processing unit is configured to generate a representation of the sensed location of the end of the catheter or tube with respect to the medical image comprising utilization of the medical image of the patient mapped to the sensor coordinate system and the location of the end of the catheter or tube in the sensor coordinate system.

In an example, the medical image is an attenuation X-ray image.

In an example, the medical image is a CT X-ray image

In an example, the medical image is a Magnetic Resonance image.

In an example, the one or more sensors are one or more cameras, and the live data stream is a live video image stream.

In an example, the one or more cameras comprises one or more 3D cameras, and the live data stream is a live video image stream or a live depth data stream.

In an example, the one or more cameras comprises one or more RGB-D cameras, and the live data stream is a live video image stream and/or a live depth data stream.

In an example, the one or more sensors are one or more lidar sensors, and the live data stream is a live lidar image stream and/or a live depth data stream.

In an example, the one or more sensors are one or more radars, and the live data stream is a live radar data stream and/or live image stream and/or live depth data stream.

In an example, the data of the patient received from the one or more sensors comprises image data of the patient received from one or more cameras.

In an example, the data of the patient received from the one or more sensors comprises lidar image data of the patient received from one or more lidar sensors.

In an example, the data of the patient received from the one or more sensors comprises lidar depth data of the patient received from one or more lidar sensors.

In an example, the data of the patient received from the one or more sensors comprises depth data of the patient received from one or more cameras.

In an example, the data of the patient received from the one or more sensors comprises depth data of the patient received from one or more radars.

In an example, the data of the patient received from the one or more sensors comprises image data of the patient received from one or more radars.

In an example, the alignment of the data of the patient with the sensor coordinate system comprises utilization of data of the patient from the one or more sensors.

Thus, knowledge of the location(s) of the one or more sensors can be used with sensor data of the patient to determine the location of the parts of the patient with respect to the location(s) of the sensors, thereby placing the patient in the sensor coordinate system.

In an example, the sensor coordinate system is a camera coordinate system.

In an example, the sensor coordinate system is a lidar sensor coordinate system.

In an example, the sensor coordinate system is a radar sensor coordinate system.

According to an example, the processing unit is configured to generate a mapped medical image of the patient comprising utilization of the medical image of the patient mapped to the sensor coordinate system. The generation of the representation of the sensed location of the end of the catheter or tube with respect to the medical image can then comprise a generation of the representation of the sensed location of the end of the catheter or tube in the mapped medical image of the patient.

In an example, the mapped medical image of the patient is a warped version of the medical image that has been transformed to align with the data of the patient from the live data stream.

In other words, the live data of the patient in the sensor coordinate system forms ground truth data and the medical image is warped to match this when presented to the medical expert along with the location of the end of the catheter or tube in the camera coordinate system.

According to an example, the processing unit is configured to generate a modified sensor coordinate system comprising utilization of the medical image of the patient mapped to the sensor coordinate system. The determination of the location of the end of the catheter or tube in the sensor coordinate system can then comprise a determination of the location of the end of the catheter or tube in the modified sensor coordinate system. The generation of the representation of the sensed location of the end of the catheter or tube with respect to the medical image can then comprise a generation of the representation of the sensed location of the end of the catheter or tube in the medical image of the patient.

According to an example, the one or more sensors comprises a plurality of sensors, and wherein the live data stream comprises data from each of the plurality of sensors.

According to an example, the alignment of the data of the patient with the sensor coordinate system comprises utilization of data of the patient from each of the plurality of sensors.

According to an example, the live data stream comprises data of the end of the catheter or tube, and wherein the sensed location of the end of the catheter or tube that is to be inserted into the patient is determined from the data of the end of the catheter or tube from each of the plurality of sensors.

In an example, the data of the end of the catheter or tube comprises image data acquired by the plurality of cameras.

In an example, the data of the end of the catheter or tube comprises depth data acquired by the plurality of cameras.

In an example, the data of the end of the catheter or tube comprises image data acquired by the plurality of lidar sensors.

In an example, the data of the end of the catheter or tube comprises depth data acquired by the plurality of lidar sensors.

In an example, the data of the end of the catheter or tube comprises image data acquired by the plurality of radars.

In an example, the data of the end of the catheter or tube comprises depth data acquired by the plurality of radars.

According to an example, a radiolocation system is configured to sense the location of the end of the catheter or tube, and wherein the radiolocation system is configured to provide the sensed location of the end of the catheter or tube to the processing unit.

According to an example, the catheter or tube comprises a fiber optic shape sensing system configured to sense the location of the end of the catheter or tube. The fiber optic shape sensing system is configured to provide the sensed location of the end of the catheter or tube to the processing unit.

According to an example, mapping the medical image of the patient to the sensor coordinate system comprises registration between the medical image of the patient and at least one frame of the live data stream.

According to an example, registration between the medical image of the patient and at least one frame of the live data stream comprises utilization of a warping transformation to align the medical image of the patient body with the data of the patient in the at least one frame of the live data stream.

FIG. 2 shows an example of a system 100 for tracing a catheter or tube. The system 100 comprises an input unit 120, a processing unit 130, one or more sensors 140, and a visual display unit 150. The input unit is configured to receive a medical image of a patient. The input unit is configured to provide the medical image of the patient to the processing unit. The input unit is configured to receive a live data stream from the one or more sensors. The live data stream comprises data of the patient, and a sensor coordinate system is defined with respect to the one or more sensors. The input unit is configured to provide the live data stream to the processing unit. The processing unit is configured to receive a sensed location of an end of a catheter or tube that is to be inserted into the patient. The processing unit is configured to align the data of the patient received from the one or more sensors with the sensor coordinate system. The processing unit is configured to map the medical image of the patient to the sensor coordinate system. The processing unit is configured to determine a location of the end of the catheter or tube in the sensor coordinate system comprising utilization of the sensed location of the end of the catheter or tube. The processing unit is configured to generate a representation of the sensed location of the end of the catheter or tube with respect to the medical image on the visual display unit comprising utilization of the medical image of the patient mapped to the sensor coordinate system and the location of the end of the catheter or tube in the sensor coordinate system.

In an example, the medical image is an attenuation X-ray image.

In an example, the medical image is a CT X-ray image

In an example, the medical image is a Magnetic Resonance image.

In an example, the one or more sensors are one or more cameras, and the live data stream is a live video image stream.

In an example, the one or more cameras comprises one or more 3D cameras, and the live data stream is a live video image stream or a live depth data stream.

In an example, the one or more cameras comprises one or more RGB-D cameras, and the live data stream is a live video image stream and/or a live depth data stream.

In an example, the one or more sensors are one or more lidar sensors, and the live data stream is a live lidar image stream and/or a live depth data stream.

In an example, the one or more sensors are one or more radars, and the live data stream is a live radar data stream and/or live image stream and/or live depth data stream.

In an example, the data of the patient received from the one or more sensors comprises image data of the patient received from one or more cameras.

In an example, the data of the patient received from the one or more sensors comprises lidar image data of the patient received from one or more lidar sensors.

In an example, the data of the patient received from the one or more sensors comprises lidar depth data of the patient received from one or more lidar sensors.

In an example, the data of the patient received from the one or more sensors comprises depth data of the patient received from one or more cameras.

In an example, the data of the patient received from the one or more sensors comprises depth data of the patient received from one or more radars.

In an example, the data of the patient received from the one or more sensors comprises image data of the patient received from one or more radars.

In an example, the alignment of the data of the patient with the sensor coordinate system comprises utilization of data of the patient from the one or more sensors.

Thus, knowledge of the location(s) of the one or more sensors can be used with sensor data of the patient to determine the location of the parts of the patient with respect to the location(s) of the sensors, thereby placing the patient in the sensor coordinate system.

In an example, the sensor coordinate system is a camera coordinate system.

In an example, the sensor coordinate system is a lidar sensor coordinate system.

In an example, the sensor coordinate system is a radar sensor coordinate system.

According to an example, the processing unit is configured to generate a mapped medical image of the patient comprising utilization of the medical image of the patient mapped to the sensor coordinate system. The processing unit is configured to present the mapped medical image on the visual display unit. The generation of the representation of the sensed location of the end of the catheter or tube with respect to the medical image can then comprise a generation of the representation of the sensed location of the end of the catheter or tube in the mapped medical image of the patient presented on the visual display unit.

According to an example, the processing unit is configured to generate a modified sensor coordinate system comprising utilization of the medical image of the patient mapped to the sensor coordinate system. The determination of the location of the end of the catheter or tube in the sensor coordinate system can then comprises a determination of the location of the end of the catheter or tube in the modified sensor coordinate system. The processing unit is configured to present the medical image on the visual display unit. The generation of the representation of the sensed location of the end of the catheter or tube with respect to the medical image can then comprise a generation of the representation of the sensed location of the end of the catheter or tube in the medical image of the patient presented on the visual display unit.

In an example, the one or more sensors comprises a plurality of sensors, and wherein the live data stream comprises data from each of the plurality of sensors.

In an example, the alignment of the data of the patient with the sensor coordinate system comprises utilization of data of the patient from each of the plurality of sensors.

In an example, the live data stream comprises data of the end of the catheter or tube, and wherein the sensed location of the end of the catheter or tube that is to be inserted into the patient is determined from the data of the end of the catheter or tube from each of the plurality of sensors.

In an example, the data of the end of the catheter or tube comprises image data acquired by the plurality of cameras.

In an example, the data of the end of the catheter or tube comprises depth data acquired by the plurality of cameras.

In an example, the data of the end of the catheter or tube comprises image data acquired by the plurality of lidar sensors.

In an example, the data of the end of the catheter or tube comprises depth data acquired by the plurality of lidar sensors.

In an example, the data of the end of the catheter or tube comprises image data acquired by the plurality of radars.

In an example, the data of the end of the catheter or tube comprises depth data acquired by the plurality of radars.

In an example, a radiolocation system is configured to sense the location of the end of the catheter or tube. The radiolocation system is configured to provide the sensed location of the end of the catheter or tube to the processing unit.

In an example, the catheter or tube comprises a fiber optic shape sensing system configured to sense the location of the end of the catheter or tube. The fiber optic shape sensing system is configured to provide the sensed location of the end of the catheter or tube to the processing unit.

In an example, mapping the medical image of the patient to the sensor coordinate system comprises registration between the medical image of the patient and at least one frame of the live data stream.

In an example, registration between the medical image of the patient and at least one frame of the live data stream comprises utilization of a warping transformation to align the medical image of the patient body with the data of the patient in the at least one frame of the live data stream.

FIG. 3 shows a method 200 for tracing a catheter or tube in its basic steps. The method 200 comprises:
receiving 210 a medical image of a patient;
providing 220 the medical image of the patient to a processing unit;
receiving 230 a live data stream from one or more sensors, wherein the live data stream comprises data of the patient, and wherein a sensor coordinate system is defined with respect to the one or more sensors;
providing 240 the live data stream to the processing unit;
receiving 250 by the processing unit a sensed location of an end of a catheter or tube that is to be inserted into the patient;
aligning 260 by the processing unit the data of the patient received from the one or more sensors with the sensor coordinate system;
mapping 270 by the processing unit the medical image of the patient to the sensor coordinate system;
determining 280 by the processing unit a location of the end of the catheter or tube in the sensor coordinate system comprising utilizing the sensed location of the end of the catheter or tube; and
generating 290 by the processing unit a representation of the sensed location of the end of the catheter or tube with respect to the medical image comprising utilizing the medical image of the patient mapped to the sensor coordinate system and the location of the end of the catheter or tube in the sensor coordinate system.

In an example, the medical image is an attenuation X-ray image.

In an example, the medical image is a CT X-ray image

In an example, the medical image is a Magnetic Resonance image.

In an example, the one or more sensors are one or more cameras, and the live data stream is a live video image stream.

In an example, the one or more cameras comprises one or more 3D cameras, and the live data stream is a live video image stream or a live depth data stream.

In an example, the one or more cameras comprises one or more RGB-D cameras, and the live data stream is a live video image stream and/or a live depth data stream.

In an example, the one or more sensors are one or more lidar sensors, and the live data stream is a live lidar image stream and/or a live depth data stream.

In an example, the one or more sensors are one or more radars, and the live data stream is a live radar data stream and/or live image stream and/or live depth data stream.

In an example, the data of the patient received from the one or more sensors comprises image data of the patient received from one or more cameras.

In an example, the data of the patient received from the one or more sensors comprises lidar image data of the patient received from one or more lidar sensors.

In an example, the data of the patient received from the one or more sensors comprises lidar depth data of the patient received from one or more lidar sensors.

In an example, the data of the patient received from the one or more sensors comprises depth data of the patient received from one or more cameras.

In an example, the data of the patient received from the one or more sensors comprises depth data of the patient received from one or more radars.

In an example, the data of the patient received from the one or more sensors comprises image data of the patient received from one or more radars.

In an example, the alignment of the data of the patient with the sensor coordinate system comprises utilization of data of the patient from the one or more sensors.

Thus, knowledge of the location(s) of the one or more sensors can be used with sensor data of the patient to determine the location of the parts of the patient with respect to the location(s) of the sensors, thereby placing the patient in the sensor coordinate system.

In an example, the sensor coordinate system is a camera coordinate system.

In an example, the sensor coordinate system is a lidar sensor coordinate system.

In an example, the sensor coordinate system is a radar sensor coordinate system.

In an example, the method comprises generating by the processing unit a mapped medical image of the patient comprising utilization of the medical image of the patient mapped to the sensor coordinate system, and the generating the representation of the sensed location of the end of the catheter or tube with respect to the medical image comprises generating the representation of the sensed location of the end of the catheter or tube in the mapped medical image of the patient.

In an example, the method comprises generating by the processing unit a modified sensor coordinate system comprising utilizing the medical image of the patient mapped to the sensor coordinate system. The determining the location of the end of the catheter or tube in the sensor coordinate system then comprises determining the location of the end of the catheter or tube in the modified sensor coordinate system, and the generating the representation of the sensed location of the end of the catheter or tube with respect to the medical image then comprises generating the representation of the sensed location of the end of the catheter or tube in the medical image of the patient.

In an example, the one or more sensors comprises a plurality of sensors, and wherein the live data stream comprises data from each of the plurality of sensors.

In an example, the aligning the data of the patient with the sensor coordinate system comprises utilizing data of the patient from each of the plurality of sensors.

In an example, the live data stream comprises data of the end of the catheter or tube, and wherein the sensed location of the end of the catheter or tube that is to be inserted into the patient is determined from the data of the end of the catheter or tube from each of the plurality of sensors.

In an example, the data of the end of the catheter or tube comprises image data acquired by the plurality of cameras.

In an example, the data of the end of the catheter or tube comprises depth data acquired by the plurality of cameras.

In an example, the data of the end of the catheter or tube comprises image data acquired by the plurality of lidar sensors.

In an example, the data of the end of the catheter or tube comprises depth data acquired by the plurality of lidar sensors.

In an example, the data of the end of the catheter or tube comprises image data acquired by the plurality of radars.

In an example, the data of the end of the catheter or tube comprises depth data acquired by the plurality of radars.

In an example, the method comprises receiving the sensed location of the end of the catheter or tube by the processing unit from a radiolocation system configured to sense the location of the end of the catheter or tube.

In an example, the method comprises receiving the sensed location of the end of the catheter or tube by the processing unit from a fiber optic shape sensing system of the catheter or tube configured to sense the location of the end of the catheter or tube the catheter or tube.

In an example, mapping the medical image of the patient to the sensor coordinate system comprises registering the medical image of the patient with at least one frame of the live data stream.

In an example, the registering the medical image of the patient with at least one frame of the live data stream comprises utilizing a warping transformation to align the medical image of the patient body with the data of the patient in the at least one frame of the live data stream.

Therefore, the new technique address provides for the automatic real-time tracking of foreign tube-shaped objects. This enables to reduce the number of incidents in the ED and ICU department relating to incorrectly inserted catheters and tubes. Thereby, it improves the overall patient wellbeing and reduces the workload of the radiologist. Furthermore, it provides direct feedback to the ED and ICU clinicians at the point of acquisition.

This is achieved by virtualizing the guidance process by real-time projection of the device being inserted to a previously acquired CXR or MR or other medical image, that could have been taken just preliminary to the insertion process of the catheter or tube or taken at a significant time-frame prior to the insertion process. The device-specific anatomical regions can also be highlighted to help to navigate to the correct insertion point

It is to be noted that in the detailed system description below, reference is made to the chest X-Ray (CXR) modality, however it is to be understood that the system is not limited to this modality, and can be applied to multiple modalities like CT, leg X-ray, and other body part and X-ray modalities, and to other medical imaging modalities such as Magnetic Resonance (MR) imaging, Positron emission tomography (PET) imaging etc. Also, reference is made below in the detailed embodiment described to a sensor system in the form of one or more cameras, but this can be a radar sensor based system, or a lidar sensor based system, or a single 3D RGB-D based system, or any other sensor based system from which data of the patient can be acquired to locate the patient within a coordinate system defined with respect to those one or more sensors.

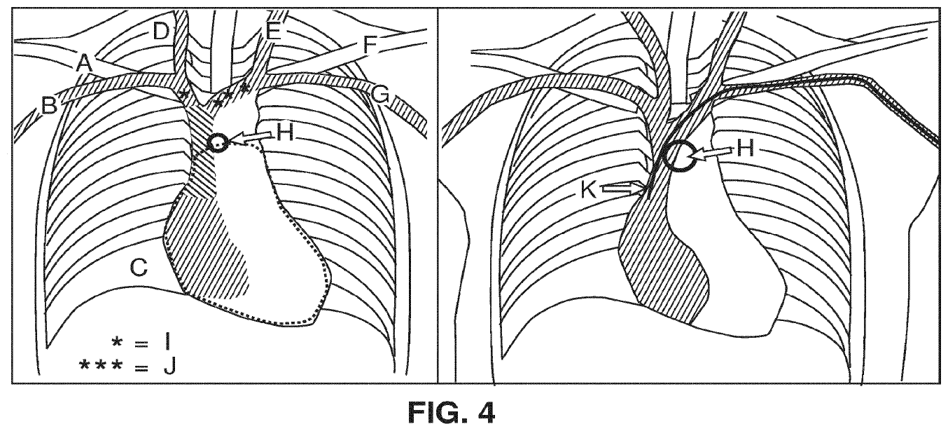

Thus with reference to FIG. 4, the position of central venous catheters and endotracheal tubes is usually assessed with respect to the anatomical point carina (=point where lower edge of left and right main bronchi meet). Also, superior vena cava (SVC) and right atrium can help to navigate radiologist to the expected region of the correct object placement. In FIG. 4 “Right clavicle” is represented by “A”, “Right subclavian vein” is represented by “B”, “pericardium” is represented by “C”, “right internal jugular vein” is represented by “D”, “left internal jugular vein” is represented by “E”, “left clavicle” is represented by “F”, “left subclavian vein” is represented by “G”, “Carina” is represented by “H”, “Right brachiocephalic vein” is represented by “T”, “left brachiocephalic vein” is represented by “J” and “Catheter tip” is represented by “K”

To help understand the new technique, an existing workflow is laid out, and the new technique is contrasted to this.

A workflow often encountered in clinical practice for the assessment of a medical image (of arbitrary modality), to rule-out of a mal-positioned foreign object, involves the following steps:
A specific tube-shaped device is inserted into the patient
A target verification examination is acquired, e.g. CXR (but could be MR etc)
The examination is sent into the PACS system and queued at the end of the worklist.
A Radiologist examines the examination after some time X
   a. If a malposition of some foreign object is detected, a report directly to clinician is made
   b. otherwise, normal reporting is carried out
   The clinician receives the report after some time Y and triggers the necessary next steps.

Both times X, Y can be long and may lead to severe problems, when the foreign object is mal-positioned with respect to the expected correct region.

The new technique described here renders steps 2-5 obsolete, because the clinician can get direct feedback in the real-time process of the device insertion. The new system can run directly on the image acquisition machine but is not limited to it. It can for example also be implemented in a cloud.

The main components of the new system are:
1) A camera for the real-time video stream (could be radar, lidar etc)
2) A target tube/catheter with a detectable tip (active/passive)
3) Antennas for triangulation and/or, radiolocation devices and/or, fiber optic integrated to catheter
4) Real-time patient body to CXR (MR etc) registration module
5) Anatomical regions segmentation/landmark detection module
6) Reporting and visualization module

In the new technique key elements of the device (the catheter/tube) are localized and that location is mapped to a previously acquired X-ray image. Below a discussion of just X-ray is made, but as discussed above this could be other medical imaging modalities such as MR. The localization can be performed via radiolocation, triangulation, fiber optic shape sensing, or other techniques known in the art. Thus, the catheter/tube tip position is localized in real-time within a patient coordinate system which is aligned with the video-stream of the patient. At the same time, a video stream is mapped to the X-ray image using an image registration algorithm. As a result, the coordinates of the tip can be presented on X-ray image.

Implementation of the automatic real-time system for tubes and catheters tracing starts with a pre-training of an image to X-ray registration model and an anatomical regions segmentation/landmark detection model. Both are deep convolutional networks of the encoder-decoder type. Both are pre-trained in advance.

In the second step, a reference image is made available to guide the procedure. This could be a pre-procedural X-ray image (e.g. a previous image of the patient) or a template/atlas image or other medical image such as MR.

The third step is the calibration of the patient-wise coordinate system with the tip and other key landmarks of the device. The patient-wise coordinate system is connected to the video-stream.

The fourth step is the inference of anatomical beacons from the anatomical region's segmentation/landmark detection models. These regions are displayed on the preliminary CXR.

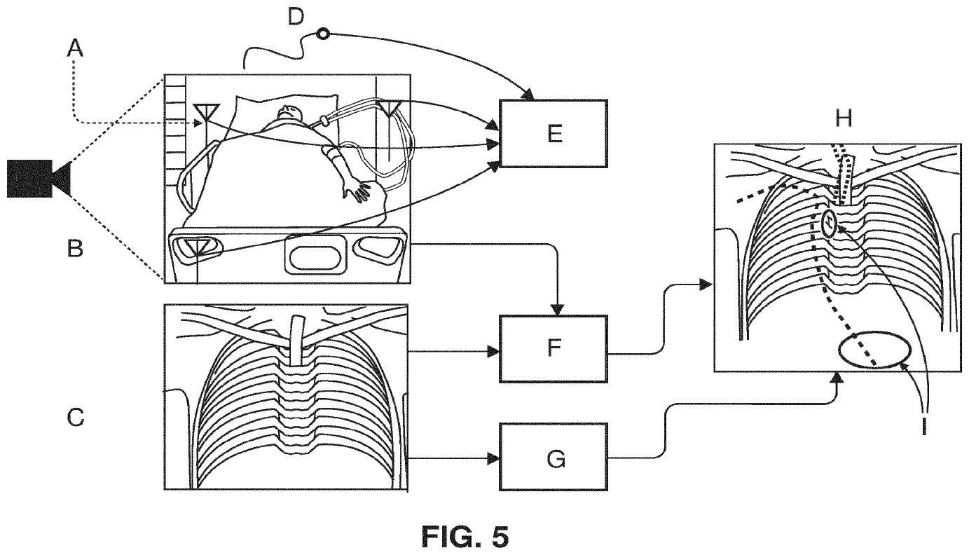

The next stage is the insertion of the tube-shaped device to the patient body (see FIG. 5). While the device is being inserted, the tip coordinates are calculated in the patient-wise coordinate system and attached to the video frames. At the same time, the video frame is registered to the preliminary taken C×R image and the patient-wise coordinate system is mapped to the CXR coordinate system. As a result, tip coordinates are converted to the CXR coordinate system. The tip coordinates are displayed on the monitor overlaying the C×R image with the anatomical beacons. The schematic overview of this process is shown in FIG. 5.

Thus, in over the new technique can be described as follows:
A patient is about to have a catheter/tube inserted into their body
An X-ray image of the patient is made available to “the system”
The system has one or more cameras that view the patient and in effect place the patient into a “camera coordinate system”
The X-ray image is mapped to the “camera coordinate system”
The X-ray imaged is mapped to the ‘camera coordinate system’ via image registration. This can be done by matching 2 images: an X-ray and a frame from the video with the patient's body. In effect a warping transformation is found that will align the patient body with the X-ray image. It can be done by neural networks or classical computer vision algorithms.
The end of the catheter/tube is monitored
This could be via the one or cameras
This could be via another system, such a radar or fiber optic based systems
The position of the catheter/tube is determined within the “camera coordinate system”
The position of the catheter/tube is therefore known with respect to the X-ray image
The position of the catheter/tube is shown with respect to the X-ray image of the patient
Thus the medical practitioner can in effect see the end of the catheter/tube with respect to an X-ray image of the patient that represents the patient in front of them, and they can see that they are going to insert the catheter/tube correctly as it approaches the patient with respect to the vein structure of the patient for example, and they can see the tip of the catheter/tube with respect to the vein structure of the patient as the catheter/tube is inserted
The medical practitioner can then correctly insert the catheter/tube into the patient.

Reference is made above to coordinate systems, however there are in fact, three coordinate systems:
1) a patient-wise coordinate system that describes the device position w.r.t. some anchors, e.g. antennas for triangulation. This coordinate system may know nothing about the patient.
2) a video-stream coordinate system that describes the patient position in time. It can be connected (calibrated) to the patient-wise coordinate system using for example the known position of antennas.
3) an X-ray coordinate system. It can be connected (calibrated) to the video-stream coordinate system via image registration.

Thus, the device's (catheter/tube) position can be viewed on X-ray. Thus in effect the video-stream is a proxy between the device and the patient.

In another exemplary embodiment, a computer program or computer program element is provided that is characterized by being configured to execute the method steps of any of the methods according to one of the preceding embodiments, on an appropriate apparatus or system.

The computer program element might therefore be stored on a computer unit, which might also be part of an embodiment. This computing unit may be configured to perform or induce performing of the steps of the method described above. Moreover, it may be configured to operate the components of the above described system. The computing unit can be configured to operate automatically and/or to execute the orders of a user. A computer program may be loaded into a working memory of a data processor. The data processor may thus be equipped to carry out the method according to one of the preceding embodiments.

This exemplary embodiment of the invention covers both, a computer program that right from the beginning uses the invention and computer program that by means of an update turns an existing program into a program that uses the invention.

Further on, the computer program element might be able to provide all necessary steps to fulfill the procedure of an exemplary embodiment of the method as described above.

According to a further exemplary embodiment of the present invention, a computer readable medium, such as a CD-ROM, USB stick or the like, is presented wherein the computer readable medium has a computer program element stored on it which computer program element is described by the preceding section.

A computer program may be stored and/or distributed on a suitable medium, such as an optical storage medium or a solid state medium supplied together with or as part of other hardware, but may also be distributed in other forms, such as via the internet or other wired or wireless telecommunication systems.

However, the computer program may also be presented over a network like the World Wide Web and can be downloaded into the working memory of a data processor from such a network. According to a further exemplary embodiment of the present invention, a medium for making a computer program element available for downloading is provided, which computer program element is arranged to perform a method according to one of the previously described embodiments of the invention.

It has to be noted that embodiments of the invention are described with reference to different subject matters. In particular, some embodiments are described with reference to method type claims whereas other embodiments are described with reference to the device type claims. However, a person skilled in the art will gather from the above and the following description that, unless otherwise notified, in addition to any combination of features belonging to one type of subject matter also any combination between features relating to different subject matters is considered to be disclosed with this application. However, all features can be combined providing synergetic effects that are more than the simple summation of the features.

While the invention has been illustrated and described in detail in the drawings and foregoing description, such illustration and description are to be considered illustrative or exemplary and not restrictive. The invention is not limited to the disclosed embodiments. Other variations to the disclosed embodiments can be understood and effected by those skilled in the art in practicing a claimed invention, from a study of the drawings, the disclosure, and the dependent claims.

In the claims, the word “comprising” does not exclude other elements or steps, and the indefinite article “a” or “an” does not exclude a plurality. A single processor or other unit may fulfill the functions of several items re-cited in the claims. The mere fact that certain measures are re-cited in mutually different dependent claims does not indicate that a combination of these measures cannot be used to advantage. Any reference signs in the claims should not be construed as limiting the scope.

1. An apparatus for tracing a catheter or a tube, comprising:
an input unit (20); and
a processor;
wherein the input unit is configured to receive a medical image of a patient;
wherein the input unit is configured to provide the medical image of the patient to the processor;
wherein the input unit is configured to receive a live data stream from one or more sensors, wherein the live data stream comprises data of the patient, and wherein a sensor coordinate system is defined with respect to the one or more sensors;
wherein the input unit is configured to provide the live data stream to the processor;
wherein the processor is configured to receive a sensed location of an end of the catheter or the tube that is to be inserted into the patient;
wherein the processor is configured to align the data of the patient received from the one or more sensors with the sensor coordinate system;
wherein the processor is configured to map the medical image of the patient to the sensor coordinate system;
wherein the processor is configured to determine a location of the end of the catheter or the tube in the sensor coordinate system comprising utilization of the sensed location of the end of the catheter or tube; and
wherein the processor is configured to generate a representation of the sensed location of the end of the catheter or the tube with respect to the medical image comprising utilization of the medical image of the patient mapped to the sensor coordinate system and the location of the end of the catheter or the tube in the sensor coordinate system.

2. The apparatus according to claim 1, wherein the processor is configured to generate a mapped medical image of the patient comprising utilization of the medical image of the patient mapped to the sensor coordinate system, and wherein the generation of the representation of the sensed location of the end of the catheter or the tube with respect to the medical image comprises a generation of the representation of the sensed location of the end of the catheter or the tube in the mapped medical image of the patient.

3. The apparatus according to claim 1, wherein the processor is configured to generate a modified sensor coordinate system comprising utilization of the medical image of the patient mapped to the sensor coordinate system, wherein the determination of the location of the end of the catheter or the tube in the sensor coordinate system comprises a determination of the location of the end of the catheter or the tube in the modified sensor coordinate system, and wherein the generation of the representation of the sensed location of the end of the catheter or the tube with respect to the medical image comprises a generation of the representation of the sensed location of the end of the catheter or the tube in the medical image of the patient.

4. The apparatus according to claim 1, wherein the one or more sensors comprises a plurality of sensors, and wherein the live data stream comprises data from each of the plurality of sensors.

5. The apparatus according to claim 4, wherein the alignment of the data of the patient with the sensor coordinate system comprises utilization of data of the patient from each of the plurality of sensors.

6. The apparatus according to claim 4, wherein the live data stream comprises data of the end of the catheter or the tube, and wherein the sensed location of the end of the catheter or the tube that is to be inserted into the patient is determined from the data of the end of the catheter or the tube from each of the plurality of sensors.

7. The apparatus according to claim 1, wherein a radiolocation system is configured to sense the location of the end of the catheter or the tube, and wherein the radiolocation system is configured to provide the sensed location of the end of the catheter or the tube to the processor.

8. The apparatus according to claim 1, wherein the catheter or the tube comprises a fiber optic shape sensing system configured to sense the location of the end of the catheter or the tube, and wherein the fiber optic shape sensing system is configured to provide the sensed location of the end of the catheter or the tube to the processor.

9. The apparatus according to claim 1, wherein mapping the medical image of the patient to the sensor coordinate system comprises registration between the medical image of the patient and at least one frame of the live data stream.

10. The apparatus according to claim 9, wherein registration between the medical image of the patient and at least one frame of the live data stream comprises utilization of a warping transformation to align the medical image of the patient body with the data of the patient in the at least one frame of the live data stream.

11. A system for tracing a catheter or a tube, comprising:
an input unit;
a processor;
one or more sensors; and
a visual display unit;
wherein the input unit is configured to receive a medical image of a patient;
wherein the input unit is configured to provide the medical image of the patient to the processor;
wherein the input unit is configured to receive a live data stream from the one or more sensors, wherein the live data stream comprises data of the patient, and wherein a sensor coordinate system is defined with respect to the one or more sensors;
wherein the input unit is configured to provide the live data stream to the processor;
wherein the processor is configured to receive a sensed location of an end of the catheter or the tube that is to be inserted into the patient;
wherein the processor is configured to align the data of the patient received from the one or more sensors with the sensor coordinate system;
wherein the processor is configured to map the medical image of the patient to the sensor coordinate system;
wherein the processor is configured to determine a location of the end of the catheter or the tube in the sensor coordinate system comprising utilization of the sensed location of the end of the catheter or the tube; and
wherein the processor is configured to generate a representation of the sensed location of the end of the catheter or the tube with respect to the medical image on the visual display comprising utilization of the medical image of the patient mapped to the sensor coordinate system and the location of the end of the catheter or the tube in the sensor coordinate system.

12. The system according to claim 11, wherein the processor is configured to generate a mapped medical image of the patient comprising utilization of the medical image of the patient mapped to the sensor coordinate system, wherein the processor is configured to present the mapped medical image on the visual display, and wherein the generation of the representation of the sensed location of the end of the catheter or the tube with respect to the medical image comprises a generation of the representation of the sensed location of the end of the catheter or the tube in the mapped medical image of the patient presented on the visual display.

13. The system according to claim 11, wherein the processor is configured to generate a modified sensor coordinate system comprising utilization of the medical image of the patient mapped to the sensor coordinate system, wherein the determination of the location of the end of the catheter or the tube in the sensor coordinate system comprises a determination of the location of the end of the catheter or the tube in the modified sensor coordinate system, wherein the processor is configured to present the medical image on the visual display, and wherein the generation of the representation of the sensed location of the end of the catheter or the tube with respect to the medical image comprises a generation of the representation of the sensed location of the end of the catheter or the tube in the medical image of the patient presented on the visual display.

14. A method for tracing a catheter or a tube, comprising:
receiving a medical image of a patient;
providing the medical image of the patient to a processor;
receiving a live data stream from one or more sensors, wherein the live data stream comprises data of the patient, and wherein a sensor coordinate system is defined with respect to the one or more sensors;
providing the live data stream to the processor;
receiving by the processor a sensed location of an end of a catheter or the tube that is to be inserted into the patient;
aligning by the processor the data of the patient received from the one or more sensors with the sensor coordinate system;
mapping by the processor the medical image of the patient to the sensor coordinate system;
determining by the processor a location of the end of the catheter or the tube in the sensor coordinate system comprising utilizing the sensed location of the end of the catheter or the tube; and
generating by the processor a representation of the sensed location of the end of the catheter or the tube with respect to the medical image comprising utilizing the medical image of the patient mapped to the sensor coordinate system and the location of the end of the catheter or the tube in the sensor coordinate system.

15. (canceled)

16. A non-transitory computer readable medium for storing executable instructions that, when executed, cause the method of claim 14 to be performed.
